(19) United States
(12) Patent Application Publication
(10) Pub. No.: US 2014/0316482 A1
(43) Pub. Date: Oct. 23, 2014

(54) MEDICAL IMPLANT HAVING A CONDUCTIVE COATING

(71) Applicant: Cardiac Pacemakers, Inc., St. Paul, MN (US)

(72) Inventors: Michael J. Kane, Roseville, MN (US); Joseph Thomas Delaney, JR., Minneapolis, MN (US)

(73) Assignee: Cardiac Pacemakers, Inc., St. Paul, MN (US)

(21) Appl. No.: 14/255,738

(22) Filed: Apr. 17, 2014

Related U.S. Application Data
(60) Provisional application No. 61/812,880, filed on Apr. 17, 2013.

(51) Int. Cl. A61L 31/10; A61L 31/02; A61L 31/14; A61N 1/362

(52) U.S. Cl. CPC A61L 31/10; A61N 1/362; A61L 31/022; A61L 31/14; A61L 2420/02; A61L 2420/08
USPC 607/36; 427/2.24

(57) ABSTRACT

A coated medical implant and associated method are disclosed. The coated medical implant can include a metallic outer surface. An intermediary layer can be coated on at least a portion of the metallic outer surface. A polyethylene glycol (PEG) self-assembled monolayer (SAM) can be formed on at least a portion of the intermediary layer. The SAM can be configured to be substantially hypoallergenic or conductive.

### CLAIM OF PRIORITY

This application claims the benefit of priority under 35 U.S.C. §119(e) of U.S. Provisional Patent Application Ser. No. 61/812,880, filed on Apr. 17, 2013, which is herein incorporated by reference in its entirety.

### TECHNICAL FIELD

This document relates generally to medical devices, and more particularly, to a medical device having a conductive portion.

### BACKGROUND

Pulse generators can be used in pacemakers, defibrillators, and other cardiac function management devices to produce an electrical discharge at desired times. Pulse generators can include pulse generation and other electronic circuitry that can be carried by a housing or “can” that can be made of a biocompatible material, such as titanium. Although titanium generally does not have a toxic or injurious effect on a patient, it can act as an allergen for patient populations. As such, pulse generator revisions are mostly attributed to pocket infections.

U.S. Pat. No. 8,175,722 is directed toward coatings for low impedance of electrodes.

Zhu, Boru, et al., “Chain-length dependence of the protein and cell resistance of oligo(ethylene glycol)-terminated self-assembled monolayers gold”, J. Biomed. Mater. Res. 2001, 56, 406-416, is directed toward forming a polymer layer on a gold substrate.

### SUMMARY

The present inventors have recognized, among other things, that titanium medical implants can cause titanium allergic reactions in a number of patients. in such instances, the allergic reaction may be deemed a pocket infection without titanium identified as an allergen. Further, certain medical implant coatings can interfere with intended or normal operation of the medical implant.

Various embodiments of the present invention can provide coatings for certain pulse generator materials. For example, titanium is a material used to manufacture pulse generator cans. The coatings described herein can be bonded to titanium. Such embodiments provide the benefit of using desired pulse generator can materials.

The pulse generator can include a hypoallergenic coating, such as a PEG coating, which can increase patient biological acceptance of the device by, for example, reducing or avoiding an allergic response to a material used to manufacture the pulse generator, such as titanium. Further, the PEG coating can reduce or avoid the occurrence of pocket infections due to allergic responses. The reduction in pocket infections can further reduce the rate of revision procedures conducted due to infections resulting from implantation or allergic response.

The present coatings can provide conductive coatings for pulse generators. A conductive coating can provide the benefit of allowing triad functionality of the pulse generator. Permitting or not interfering with triad configurations can increase the functionality or reliability of implanted pulse generators.

The coatings described herein can include economical pulse generator coatings. For example, PEG can be used to coat a pulse generator. PEG is a readily available, inexpensive material that can be bonded to a pulse generator can to provide a hypoallergenic conductive coating.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 is a perspective view of a medical implant;

FIG. 2 is a perspective view of a coated medical implant;

FIG. 3 is a perspective view of a medical implant with a self-assembled monolayer; and

FIG. 4 is a method of coating a medical implant.

### DETAILED DESCRIPTION

The present disclosure describes, among other things, a substantially hypoallergenic medical implant and related method. The medical implant can include metallic outer surface, an intermediary layer coated on at least a portion of the metallic outer surface, and a polyethylene glycol (PEG) self-assembled monolayer (SAM) formed on at least a portion of the intermediary layer. The SAM can be configured to be substantially hypoallergenic or conductive.

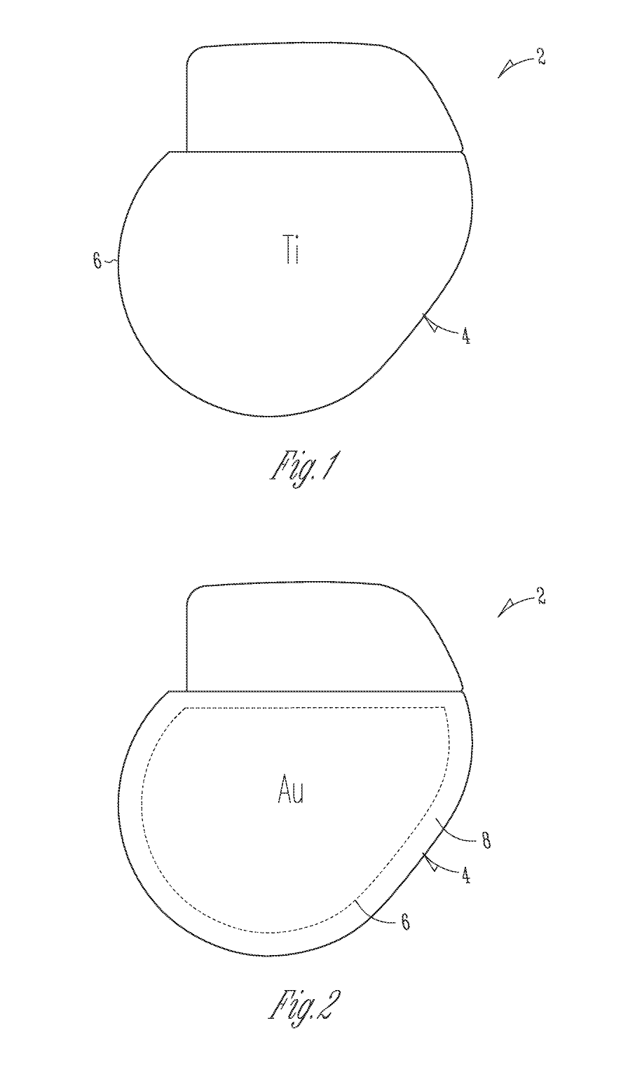

FIG. 1 is a perspective view of a medical implant 2, The medical implant 2 can include a pulse generator housing or “can,” such as can be used in a cardiac or other electrostimulation device to house one or more components and optionally to use or convey an electric pulse. The medical implant 2 can include an outer surface 4, such as can include a metallic material 6. The metallic material 6 can include a biocompatible material, such as stainless steel, gold, silver, cobalt-chromium, titanium-based, or one or more combinations thereof.

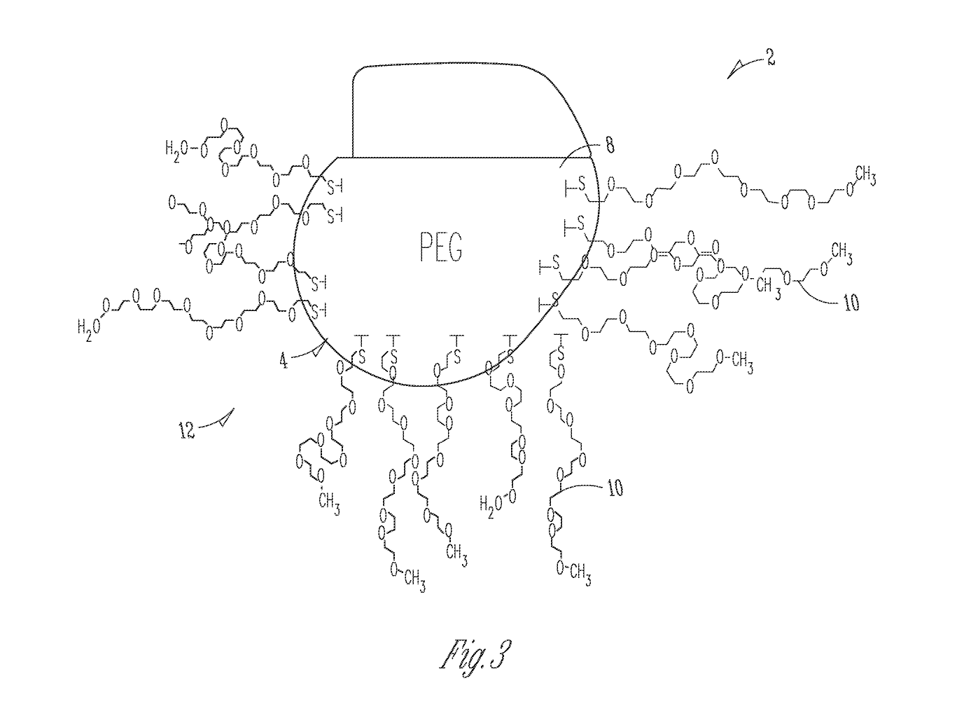

FIG. 2 is a perspective view of a coated medical implant 2. The outer surface 4 can at least be partially coated with an intermediary layer 8. The intermediary layer can include gold, such as an epitaxial layer of gold. The intermediary layer 8 can be sputtered, deposited, such as by chemical vapor deposition, or electrochemically adhered to the metallic material 6, such that a substantially even coating or exclusively adhered coating on the electrode surface, such as the titanium outer surface 4, such that gold can be adhered to at least a portion of substantially all areas of the electrode that are accessible by current, for example, and gold atoms in solution.

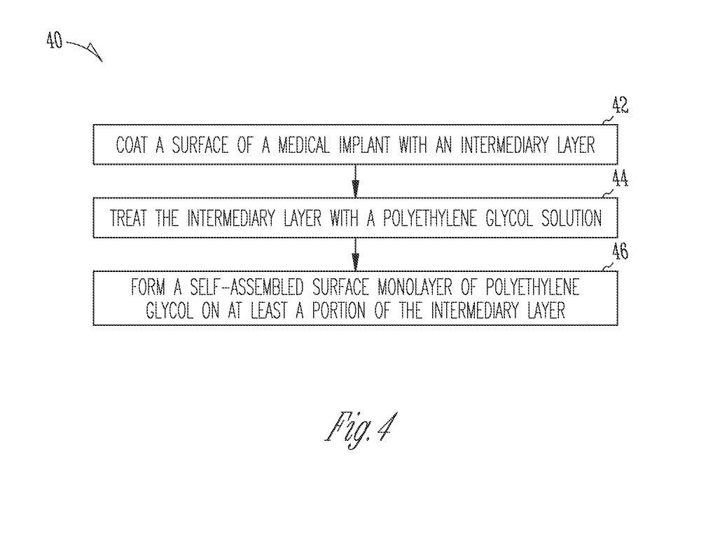

FIG. 3 is a perspective view of a medical implant 2 that can include a substantially hypoallergenic self-assembled monolayer (SAM) 12. Substantially hypoallergenic includes substantially preventing an allergic reaction of a patient to an implanted medical implant. One way to determine whether a medical implant is substantially hypoallergenic is to administer a skin test, such as a patch test, to a patient to determine whether an allergic reaction occurs when the patient is exposed to the uncoated material of the implant, and then to expose that patient to the coated material if no allergic reaction to the coated material is observed, the material can be declared hypoallergenic for that patient. A similar test can be carried out on a population of human or animal test subjects. If this difference in results is observed for more than a specified proportion of test subjects (e.g., for more than 80 percent of the test subjects), then the coating can be regarded as substantially hypoallergenic. Another hypoallergenic, hypersensitivity, or intolerance test can include an in vitro test, such as with the lymphocyte transformation test (LTT) or memory lymphocyte immunostimulation assay (e.g., MELISA®).

The SAM 12 can include a polymer such as polyethylene glycol (PEG), for example, covalently bonded PEG chains 10 with the intermediary layer 8. The PEG can include commercially available PEG of varying molecular weight, including, but not limited to, from about PEG 600 to about PEG 10,000. The PEG can be terminated in a functional group, such as thiol terminated, so that a gold-thiol bond can be formed. between an individual PEG chain 10 and the gold layer 8. The bond between the PEG chain 10 and the gold layer 8 can provide a bond strength of at least about 85 kJ/mol.

By depositing an epitaxial layer of gold that creates the gold intermediary layer 8 and then treating the gold intermediary layer 8 with thiol-terminated PEG chains 10, the SAM 12 can be made that can exhibit one or more stealth effects. As used herein, “stealth effects” refer to the ability of the SAM 12 to effectively ‘hide’ the metallic outer material 4, such that an immune response, such as an allergic reaction to the metallic outer material 4, can be substantially avoided. By including the SAM 12, the medical implant 2 can be produced without an insulating medium of, for example, parylene or silicone rubber.

In addition, the SAM 12 can inhibit non-specific protein binding and cell adhesion, thus inhibiting or preventing one or more of bio-fouling or tissue ingrowth. Inhibiting bio-fouling or tissue ingrowth can reduce an accumulation of proteins or tissue on the medical implant 2, outer surface 4, gold intermediary layer 8, or SAM 12, which can be beneficial since the accumulation of proteins or tissue can increase impedance of implanted electrodes, for example, the medical implant 2, consequently increasing power consumption and efficiency. Additional benefits of the present SAM 12 can include one or more of improved wettability, lower impedance, or resistance to contamination.

Further, additional stealth effects provided by the SAM 12 are enabled by the internal hydrophilicity or terminal hydrophilicity of the SAM 12 when implanted in a patient's body. For example, the SAM 12 can present to the patient's body a layer of water, such that the SAM 12 can be generally undetected by the patient's body. For example, the SAM 12 can be a non-continuous coating of PEG where the PEG chains 10 do not interfere with one another, while also providing a coating that substantially encases the entire outer surface 4. The properties of the PEG chains 10 can provide for this result. For example, as a result of the chain architecture and spacing between oxygen atoms, the PEG chains 10 can hydrogen bond with water, forming a three dimensionally continuous shell around the PEG chains 10. When the PEG chains 10 are completely surrounded by water, they can pass unrecognized by proteins. By grafting the PEG chains 10 to the outer surface 4, this unique property can be imparted on the surface 4, and the medical implant can be configured to have less bio-fouling, as compared to a medical device without the SAM 12.

In some embodiments, the SAM 12 can allow the medical implant 2 to remain an active electrode in a TRIAD defibrillation configuration. In a dual configuration, a shock or defibrillation event occurs between two electrodes, the distal and proximal coil electrodes. However, in the TRIAD configuration, the shock or defibrillation event occurs between the distal coil electrode, the proximal coil electrode, and the pulse generator can 2. As a result, the pulse generator can 2 can be capable of operating in a greater variety of configurations while still maintaining a hypoallergenic coating, such as SAM 12.

FIG. 4 is an example of a method 40 of coating a medical implant. In describing the method 40 reference is made to features and elements previously described herein, including numbered references. Numbered elements provided within the description of the method 40 are not intended to be limiting, instead numbered references are provided for convenience and can include any similar features described herein, as well as their equivalents. The method 40 can include coating 42 a surface 4 of a medical implant 2 with an intermediary layer 8. Coating 42 can include at least one of sputtering, chemical vapor depositing, or electrochemical processing, such as described herein. For example, coating 42 can include coating with an intermediary layer 8 of gold.

The method 40 can include treating 44 the intermediary layer 8 with a PEG solution. Such a solution can include at least one of ethanol and thiol-terminated PEG. For example, the medical implant 2 can be dipped in the PEG solution, such that the intermediary layer 8 can be immersed in the PEG solution, such as for at least about 20 minutes. Further, the PEG solution can include a concentration of at least about 0.1 (mM) PEG in ethanol.

The method 40 can include forming 46 a SAM 12 of PEG on at least a portion of the intermediary layer 8, such as by PEGylation, including a process of covalent attachment of PEG chains to another molecule, such as a surface of an object. The SAM 12 can be configured to be substantially hypoallergenic, such as described herein. For example, forming 46 the SAM 12 can include forming gold-thiol bonds between the individual PEG chains 10 and the gold intermediary layer 8.

Further, the method 40 can include rinsing the medical implant 2, after forming the SAM, such as rinsing with substantially pure ethanol and drying the pulse generator in a substantially nitrogen environment, such as in a heater or oven.

Surface-based gelation chemistry can be used, such as where gold-thiol chemistry can be used to bond one of the components for a gel layer to form, followed by a second, separate, orthogonal chemical reaction enabling gelation. An example of this can include surface modification such as using a thiol-functionalized derivative of polyethyleneinine or thiol-functionalized chitosan, which can be allowed to subsequently react with a solution of maleimide-terminated PEG chains. If the chains are monofunctional, a brush system can be generated; if the PEG chains are telechelically bifunctional, a gel network can form instead, such as can offer additional durability.

A chemically orthogonal reaction that can be employed, such as including the Huisgen [3±2] cycloaddition reaction, in which partially thiol-functionalized poly(vinyl alcohol-co-propargyl ether) can be allowed to react with a gold surface, such that azide-terminated PEG can be added, along with ascorbic acid and a copper catalyst, to promote the intended clicking reaction. Diels-Alder cycloaddition and/or coupling reactions, through amide or ester bond formation, can be used for forming the SAM 12. If monofunctional PEG is used, a brush system can result, while a telechelic bisfunctional diazide PEG can yield a crosslinked network.

A complexing ionic system can be used to gelate on the surface. As an example, thiol-functionalized heparin can be used to coat the gold, which can then be treated with PEGylated chitosan or PEGylated polyethyleneimine, to yield an ionically crosslinked gel system. Such a layer-by-layer strategy can be readily repeated sequentially to build up thicker layers of gel, if so desired. This represents an example of PEGylation involving linear chain architecture, which can be facile to employ. However, other macromolecular architectures employing PEG can also be suitable, including one or more of stars, brushes, or combs. Chemically bonded PEG gels can be used in a similar fashion, with PEG thiol bonded to the surface offering good mechanical stability, with the other chain ends branching off into networks; this can offer the advantage of a controllable, rugged, thick coating of PEG, but may be more difficult to implement.

Further examples of PEGylation techniques of titanium can include a two-step treatment of using a polyisocyanate-functionalized primer, such as 4,4′-MDI, on the outer surface 4, followed by treating the primer with a PEG solution that is hydroxyl-terminated, amine-terminated, or any other appropriately isocyanate-reactive function group terminated. The outer surface 4 can be treated with a PEG solution that can be end-functionalized or terminated with one or more phosphate or phosphate esters. Then, for example, the outer surface 4 on the medical implant 2 can be annealed at a temperature of at least about 100° C. Annealing can react the passivated oxide to form an inorganic ester bond between the PEG and outer surface material 6. Further, a sianization process can be used to form the SAM 12. For example, the outer surface 4 can be treated using one or more silanyl chlorides or ortho-esters, followed by treatment with hydroxyl-terminated PEG, or the functionalization of PEG with one or more silanyl chlorides or ortho-silane esters, followed by deposition on the outer surface 4.

### EXAMPLES

### Impedance Testing

A PEGylated device, according to an example of the present disclosure, and a non-PEGylated control device, Endotak Reliance® Implantable Lead serial number 0158-305381, were tested. The two devices were subjected to 70 simulated defibrillation pulse cycles at 700 volts (V) and 15 Amps (A) for 18 milliseconds (ms). Each defibrillation electrode coil was placed in a phosphate buffered saline solution in a 1 liter volumetric flask. The impedance values for 2.5 the first five and last five cycles of each coil were recorded. The results of those 10 cycles are listed in Table 1.

TABLE 1
Impedance Results

| | |
|---|---|
| PEGylated Device | Control Device |

| | First Five | Last Five | First Five | Last Five |
|---|---|---|---|---|
| | Shocks | Shocks | Shocks | Shocks |
| | (Ω) | (Ω) | (Ω) | (Ω) |
| | 35.71 | 35.23 | 36.29 | 36.99 |
| | 35.74 | 34.47 | 35.45 | 37.02 |
| | 35.21 | 35.22 | 36.26 | 37.01 |
| | 35.70 | 35.22 | 36.29 | 36.90 |
| | 35.11 | 35.27 | 36.23 | 36.04 |
| Average | 35.49 | 35.08 | 36.10 | 36.79 |
| Standard | 0.31 | 0.34 | 0.37 | 0.42 |
| Deviation | | | | |

The results in Table 1 indicate that PEGylation of a device has no negative impact on defibrillation electrode impedance, and can have positive impact. Further, the PEG coating on the device was shown to be stable after 70 defibrillation pulse cycles. A T-test of the last five shocks on the PEGylated device resulted in 5.5×10⁻⁵, indicating a statistically significant difference.

### ADDITIONAL NOTES AND EXAMPLES

Example 1 can include subject matter (such as an apparatus, a method, a tangible non-transitory device-readable medium for performing all or a portion of a method, or a means for performing certain acts) that can include or use a medical implant, comprising a metallic surface, an intermediary layer coated on at least a portion of the metallic surface, and a polyethylene glycol (PEG) self-assembled monolayer (SAM) formed on at least a portion of the intermediary layer.

Example 2 can include, or can optionally be combined with the subject matter of Example 1, to optionally include wherein the medical implant is a pulse generator can, and wherein the SAM is configured to permit a triad shock configuration of the pulse generator can.

Example 3 can include, or can optionally be combined with the subject matter of one or any combination of Examples 1 or 2, to optionally include wherein the PEG is terminated in at least one functional group configured to bond with the intermediary layer.

Example 4 can include, or can optionally be combined with the subject matter of one or any combination of Examples 1-3, to optionally include wherein the intermediary layer at least includes gold.

Example 5 can include, or can optionally be combined with the subject matter of one or any combination of Examples 1-4, to optionally include wherein the PEG SAM is covalently bonded to the intermediary layer, including a gold-thiol bond.

Example 6 can include, or can optionally be combined with the subject matter of one or any combination of Examples 1-5, to optionally include wherein the PEG SAM is configured to be a protein resistant surface.

Example 7 can include, or can optionally be combined with the subject matter of one or any combination of Examples 1-6, to optionally include wherein a bond between the intermediary layer and the SAM is at least about 85 kJ·mol⁻¹.

Example 8 can include, or can optionally be combined with the subject matter of one or any combination of Examples 1-7, to optionally include wherein the metallic outer surfaces includes at least titanium.

Example 9 can include, or can optionally be combined with the subject matter of one or any combination of Examples 1-8, to optionally include wherein a density of the PEG SAM is configured to substantially prevent bio-fouling of the pulse generator can.

Example 10 can include, or can optionally be combined with the subject matter of one or any combination of Examples 1-9, to optionally include wherein the SAM is configured to be conductive.

Example 11 can include subject matter (such as an apparatus, a method, a tangible non-transitory device-readable medium for performing all or a portion of a method, or a means for performing certain acts) that can include coating a surface of a medical implant with an intermediary layer, treating the intermediary layer with a polyethylene glycol (PEG) solution, and forming a self-assembled surface monolayer (SAM) of PEG on at least a portion of the intermediary layer.

Example 12 can include, or can optionally be combined with the subject matter of Example 11, to optionally include wherein coating includes coating the surface with gold and the forming includes forming gold-thiol bonds between the gold intermediary layer and the PEG, wherein the PEG is terminated in at least one functional group configured to bond with the intermediary layer.

Example 13 can include, or can optionally be combined with the subject matter of one or any combination of Examples 11 or 12, to optionally include wherein coating the surface includes at least one of sputtering, chemical vapor depositing, and electrochemical processing.

Example 14 can include, or can optionally be combined with the subject matter of one or any combination of Examples 11-13, to optionally include wherein forming the SAM includes PEGylation.

Example 15 can include, or can optionally be combined with the subject matter of one or any combination of Examples 11-14, to optionally include wherein treating the intermediary layer includes at least partially immersing the intermediary layer in the PEG solution for at least about 20 minutes.

Example 16 can include, or can optionally be combined with the subject matter of one or any combination of Examples 11-15, to optionally include wherein forming the SAM includes covalently bonding PEG to the intermediary layer.

Example 17 can include, or can optionally be combined with the subject matter of one or any combination of Examples 11-16, to optionally include wherein treating the intermediary layer includes immersing the intermediary coated surface in a solution having a concentration of at least about 0.1 millimole (mM) PEG.

Example 18 can include, or can optionally be combined with the subject matter of one or any combination of Examples 11-17, to optionally include further comprising, after forming the SAM of PEG, rinsing the medical implant with ethanol and drying the medical implant in a nitrogen environment.

Example 19 can include, or can optionally be combined with the subject matter of one or any combination of Examples 11-18, to optionally include wherein treating the intermediary layer includes using the PEG solution including at least one of ethanol and thiol-terminated PEG.

Example 20 can include subject matter (such as an apparatus, a method, a tangible non-transitory device-readable medium for performing all or a portion of a method, or a means for performing certain acts) that can include or use a medical implant pulse generator can, comprising: a titanium surface, a gold intermediary layer coated on at least a portion of the titanium outer surface, and a polyethylene glycol (PEG) self-assembled monolayer (SAM) formed on at least a portion of the gold intermediary layer, wherein the SAM includes a plurality of independent gold-thiol bonds between the gold intermediary layer and the PEG, wherein the SAM is configured to be substantially hypoallergenic, wherein the SAM is configured to permit a triad shock configuration of the pulse generator can.

Example 21 can include, or can optionally be combined with any portion or combination of portions of any one or more of Examples 1-20 to include, subject matter that can include means for performing any one or more of the functions of Examples 1-20, or a machine readable medium including instructions that, when performed by a machine, cause the machine to perform any one or more of the functions of Examples 1-20.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods, The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A medical implant, comprising:
a metallic surface;
an intermediary layer coated on at least a portion of the metallic surface; and
a polyethylene glycol (PEG) self-assembled monolayer (SAM) formed on at least a portion of the intermediary layer.

2. The medical implant of claim 1, wherein the medical implant is a pulse generator can, and wherein the SAM is configured to permit a triad shock configuration of the pulse generator can.

3. The medical implant of claim 1, wherein the PEG is terminated in at least one functional group configured to bond with the intermediary layer.

4. The medical implant of claim 1, wherein the intermediary layer at least includes gold.

5. The medical implant of claim 4, wherein the PEG SAM is covalently bonded to the intermediary layer, including a gold-thiol bond.

6. The medical implant of claim 1, wherein the PEG SAM is configured to be a protein resistant surface.

7. The medical implant of claim 1, wherein a bond between the intermediary layer and the SAM is at least about 85 kJ·mol⁻¹.

8. The medical implant of claim 1, wherein the metallic outer surfaces includes at least titanium.

9. The medical implant of claim 1, wherein a density of the PEG SAM is configured to substantially prevent bio-fouling of the pulse generator can.

10. The medical implant of claim 1, wherein the SAM is configured to be conductive.

11. A method of coating a medical implant, the method comprising:
coating a surface of a medical implant with an intermediary layer;
treating the intermediary layer with a polyethylene glycol (PEG) solution; and
forming a self-assembled surface monolayer (SAM) of PEG on at least a portion of the intermediary layer.

12. The method of claim 11, wherein coating includes coating the surface with gold and the forming includes forming gold-thiol bonds between the gold intermediary layer and the PEG, wherein the PEG is terminated in at least one functional group configured to bond with the intermediary layer.

13. The method of claim 11, wherein coating the surface includes at least one of sputtering, chemical vapor depositing, and electrochemical processing.

14. The method of claim 11, wherein forming the SAM includes PEGylation.

15. The method of claim 11, wherein treating the intermediary layer includes at least partially immersing the intermediary layer in the PEG solution for at least about 20 minutes.

16. The method of claim 11, wherein forming the SAM includes covalently bonding PEG to the intermediary layer.

17. The method of claim 11, wherein treating the intermediary layer includes immersing the intermediary coated surface in a solution having a concentration of at least about 0.1 millimole (mM) PEG.

18. The method of claim 11, further comprising, after forming the SAM of PEG, rinsing the medical implant with ethanol and drying the medical implant in a nitrogen environment.

19. The method of claim 1, wherein treating the intermediary layer includes using the PEG solution including at least one of ethanol and thiol-terminated PEG.

20. A medical implant pulse generator can, comprising:
a titanium surface;
a gold intermediary layer coated on at least a portion of the titanium outer surface; and
a polyethylene glycol (PEG) self-assembled monolayer (SAM) formed on at least a portion of the gold intermediary layer, wherein the SAM includes a plurality of independent gold-thiol bonds between the gold intermediary layer and the PEG, wherein the SAM is configured to be substantially hypoallergenic, wherein the SAM is configured to permit a triad shock configuration of the pulse generator can.
